(19) United States
(12) Patent Application Publication
(10) Pub. No.: US 2026/0026826 A1
(43) Pub. Date: Jan. 29, 2026

(54) INTRODUCER LOCK APPARATUS

(71) Applicant: Boston Scientific Scimed, Inc., Maple Grove, MN (US)

(72) Inventors: Michael Turovskiy, San Leandro, CA (US); Roy Leguidleguid, Union City, CA (US); Alan Thomas, Pleasanton, CA (US); Jayson Delos Santos, Alamo, CA (US)

(73) Assignee: Boston Scientific Scimed, Inc., Maple Grove, MN (US)

(21) Appl. No.: 18/993,415

(22) PCT Filed: Jul. 11, 2023
(86) PCT No.: PCT/US2023/027332

§ 371 Date: Jan. 10, 2025

Related U.S. Application Data
(60) Provisional application No. 63/388,037, filed on Jul. 11, 2022.

(51) Int. Cl. A61B 17/221; A61B 17/00; A61M 25/00

(52) U.S. Cl. CPC A61B 17/221; A61B 2017/00292; A61M 2025/0058

(57) ABSTRACT

Medical devices and methods for using medical devices are disclosed. An example medical device lock for use with a thrombectomy system includes a body having a lumen extending therein, a first clamping arm pivotably coupled to the body and a second clamping arm pivotably coupled to the body. Additionally, the first clamping arm may be releasably attached to the second clamping arm to clamp a catheter shaft therebetween, wherein clamping the catheter between the first clamping arm and the second clamping arm maintains the catheter shaft in a substantially linear configuration.

### CROSS-REFERENCE TO RELATED APPLICATIONS

This application claims the benefit of priority of U.S. Provisional Application No. 63/388,037, filed Jul. 11, 2022, the entire disclosure of which is hereby incorporated by reference.

### BACKGROUND

Minimally invasive medical procedures may be preferred by clinicians and patients because of a faster healing time and decreased pain. For example, minimally invasive procedures that use a catheter assembly may need only small incisions to enter the body, blood vessel, or other body lumen. Small incisions may be less painful and heal more rapidly than larger incisions, especially large incisions that open up major portions of the patient's body.

A catheter may be guided to a treatment area through one or more small incisions and medical treatment provided. Following treatment, the catheter may be withdrawn, and any incisions closed. As part of the medical procedure, a distal end of the catheter may be coupled to tools that may be controlled by one or more wires, shafts, or tendons within the catheter. In some cases, manipulation of the wires, shafts, or tendons may require moving them relative to an outer catheter.

In particular, devices for mechanically removing material, including thrombus material, from within a lumen of the vessel may include a catheter over which an inverting tube may be used to remove material from within the body lumen. For example, these devices may be used as mechanical thrombectomy catheters for removing a clot from a blood vessel (e.g., thrombectomy devices), similar to those disclosed and described in each of U.S. Pat. Nos. 10,271,864, 10,835,234, 10,561,431, 10,842,513 and 11,253,291. These apparatuses do an excellent job at removing material from within a blood vessel but may require multiple hands to operate.

Thus, there is a need for devices and systems including these devices, that can be used to help control catheters, an in particular catheters in which a flexible tube is inverted and rolled over or at the distal end of the catheter, to enhance the ease of use and precision with which these apparatuses may be operated. Described herein are apparatuses (devices, systems and kit) and methods of using them that may address the needs and problems discussed above.

### SUMMARY OF THE DISCLOSURE

This disclosure provides design, material, manufacturing method, and use alternatives for medical devices. An example medical device lock for use with a thrombectomy system includes a body having a lumen extending therein, a first clamping arm pivotably coupled to the body and a second clamping arm pivotably coupled to the body. Additionally, the first clamping arm may be releasably attached to the second clamping arm to clamp a catheter shaft therebetween, wherein clamping the catheter between the first clamping arm and the second clamping arm maintains the catheter shaft in a substantially linear configuration.

Alternatively or additionally to any of the embodiments above, wherein the first clamping arm is coupled to the body via a first hinge connection.

Alternatively or additionally to any of the embodiments above, wherein the second clamping arm is coupled to the body via a second hinge connection.

Alternatively or additionally to any of the embodiments above, wherein the first hinge connection includes a first spring configured to bias the first clamping arm in an unlocked configuration.

Alternatively or additionally to any of the embodiments above, wherein the second hinge connection includes a second spring configured to bias the second clamping arm in the unlocked configuration.

Alternatively or additionally to any of the embodiments above, wherein the lumen of the body is configured to permit the catheter shaft to extend therethrough.

Alternatively or additionally to any of the embodiments above, wherein the first clamping arm includes a first clamping channel extending along a longitudinal axis of the first clamping arm, wherein the first clamping channel is configured to permit the catheter shaft to be disposed therein.

Alternatively or additionally to any of the embodiments above, wherein the second clamping arm includes a second clamping channel extending along a longitudinal axis of the second clamping arm, wherein the second clamping channel is configured to permit the catheter shaft to be disposed therein.

Alternatively or additionally to any of the embodiments above, wherein the first clamping channel, the second clamping channel or both the first clamping channel and the second clamping channel includes a silicone pad disposed thereon.

Alternatively or additionally to any of the embodiments above, wherein the first clamping arm includes a locking member disposed along a proximal end region of the first clamping arm, and wherein the locking member is configured to releasably engage a portion of the second clamping arm.

Alternatively or additionally to any of the embodiments above, wherein the second clamping arm includes a cradle disposed along a proximal end region of the second clamping arm, wherein the cradle is configured to accept the catheter shaft.

Alternatively or additionally to any of the embodiments above, wherein the cradle is longitudinally aligned with the lumen of the attachment body.

Alternatively or additionally to any of the embodiments above, further comprising a connector disposed along a distal end region of the body.

Alternatively or additionally to any of the embodiments above, wherein the connector is configured to attach to a manifold of a medical introducer sheath.

Alternatively or additionally to any of the embodiments above, wherein releasably attaching the first clamping arm to the second clamping arm to clamp a catheter shaft therebetween prevents the catheter shaft from translating relative to the manifold of the introducer sheath.

Alternatively or additionally to any of the embodiments above, wherein the catheter shaft is an outer shaft of a thrombectomy apparatus.

A medical device lock for use with a thrombectomy system includes a body having a distal end region, a proximal end region and a lumen extending therein. Further, the medical device includes a first clamping arm coupled to the proximal end region of the body, wherein the first clamping arm is configured to pivot about a first spring-loaded hinge connection and a second clamping arm coupled to the proximal end region of the body, wherein the second clamping arm is configured to pivot about a second spring-loaded hinge connection. Additionally, the first clamping arm may be releasably attached to the second clamping arm to clamp a catheter shaft between the first clamping arm and the second clamping arm in a locked configuration, and wherein clamping the catheter between the first clamping arm and the second clamping arm maintains the shaft in a substantially linear configuration.

Alternatively or additionally to any of the embodiments above, wherein the first hinge connection includes a first spring configured to bias the first clamping arm in an unlocked configuration, and wherein the second hinge connection includes a second spring configured to bias the second clamping arm in the unlocked configuration.

Alternatively or additionally to any of the embodiments above, wherein the first clamping arm includes a locking member disposed along a proximal end region of the first clamping arm, and wherein the locking member is configured to releasably engage a portion of the second clamping arm in the locked configuration.

An example method of securing a portion of a thrombectomy system relative to an introducer includes positioning the introducer within a body vessel of a patient, the introducer including a shaft coupled to a manifold. The method also includes securing a medical device lock to the manifold of the introducer. The medical device lock includes a body having a lumen extending therein, a first clamping arm pivotably coupled to the body and a second clamping arm pivotably coupled to the body. Additionally, the first clamping arm may be releasably attached to the second clamping arm. Additionally, the method may further include advancing an outer catheter of the thrombectomy system through the lumen of the medical device lock body, through the manifold of the introducer and into the lumen of the introducer. The method may also include attaching the first clamping arm to the second clamping arm such that the outer catheter is clamped between the first clamping arm and the second clamping arm, and wherein clamping the outer shaft between the first clamping arm and the second clamping arm prevents the outer catheter from translating relative to the introducer.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 illustrates an example of a system including a mechanical thrombectomy apparatus (e.g., outer catheter, inverting tube, and puller, along with a guidewire), as well as an introducer sheath and an introducer lock apparatus as described herein. The system is shown within a blood vessel proximate to a clot.

FIGS. 2A-2C illustrate an example of a mechanical thrombectomy apparatus as described herein, including an inverting tube that may be used to remove material from a vessel. In FIG. 2A, the assembled apparatus is shown in a side view, showing an inversion support catheter and a flexible outer tube. FIG. 2B shows the inverting tube apparatus of FIG. 2A in a vessel, proximal to a clot. FIG. 2C illustrates the removal of a clot from the vessel using the apparatus of FIG. 2A, by pulling the flexible tube on the outside of the inversion support catheter proximally so that it rolls over the distal end of the inversion support catheter and into the inversion support catheter, drawing the clot with it; the apparatus may be advanced distally.

FIG. 3A shows a first view of a catheter controlling system, in accordance with some examples.

FIG. 3B shows a second view of the catheter controlling system, in accordance with some examples.

FIG. 4A shows a cross sectional view of a catheter that may include an inner shaft.

FIG. 4B shows a side view of a catheter, in accordance with some examples.

FIG. 5A shows a first view of a catheter controlling system, in accordance with some examples.

FIG. 5B shows a second view of the catheter controlling system.

FIG. 6A shows a first view of an introducer lock apparatus, in accordance with some examples.

FIG. 6B shows a second view of the introducer lock apparatus, in accordance with some examples.

FIG. 6C shows a third view of the introducer lock apparatus, in accordance with some examples.

FIG. 7A shows a first view of a first body of the introducer lock apparatus of FIGS. 6A-6C.

FIG. 7B shows a second view of the first body of the introducer lock apparatus of FIGS. 6A-6C.

FIG. 7C shows a third view of the first body of the introducer lock apparatus of FIGS. 6A-6C.

FIG. 8A shows a first view of a second body of the catheter retainer lock of FIG. 6A-6C.

FIG. 8B shows a second view of the second body of the catheter retainer lock of FIGS. 6A-6C.

FIG. 8C shows a third view of the second body of the catheter retainer lock of FIGS. 6A-6C.

FIG. 9 is a flowchart depicting an example of one method for controlling a catheter;

FIG. 10 shows a perspective view of another introducer lock apparatus;

FIG. 11 shows another perspective view of the introducer lock apparatus of FIG. 10;

FIG. 12 shows the introducer of FIGS. 10-11 in accordance with some examples;

FIG. 13 shows the introducer of FIGS. 10-11 in accordance with some examples.

### DETAILED DESCRIPTION

Minimally invasive medical procedures often use catheter-based systems to provide therapy that would otherwise require more traumatic methods. The smaller incisions associated with catheter therapies that are used to enter the body may promote faster healing and lessen the recovery time for the patient. During minimally invasive procedures, a distal end of the catheter may be used to manipulate various tools or internal organs while the clinician operates at a distance. In some cases, the clinician may use robotics to manipulate or control the catheter.

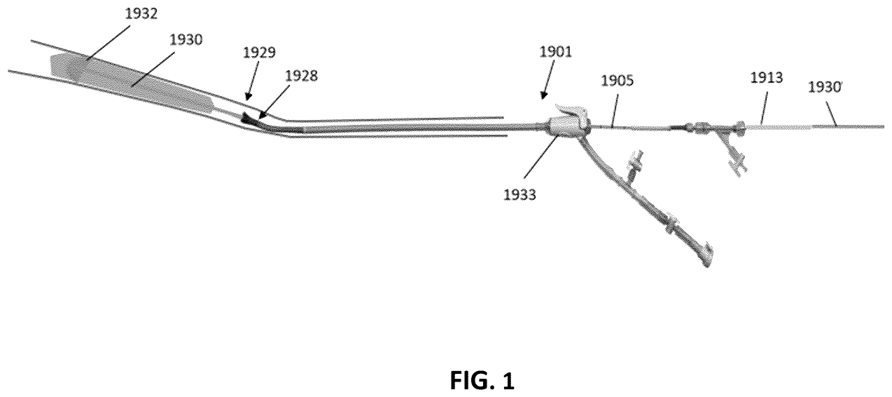

FIG. 1 illustrates one example of a system including both a mechanical thrombectomy apparatus 1901 (also referred to herein as an inverting thrombectomy apparatus) an introducer 1933. In FIG. 1, the mechanical thrombectomy apparatus 1901 is shown deployed within a vessel proximate to a clot 1930, 1930′. In this example, an optional expandable scraper 1932 is shown deployed through the inverting thrombectomy apparatus and into the clot, and expanded within the clot, as described above. The inverting thrombectomy apparatus generally includes any of the features discussed above, including an inversion support catheter, a flexible (e.g., knitted) tube 1928 inverted over the distal end 1929 of the inversion support catheter, and a puller 1913. In this example, an optional expandable scraper 1932 is deployed through the inverting thrombectomy apparatus (e.g., through the flexible tube and inversion support catheter) and includes a distal expanding (scraping) region (“distal expanding region”) 1932. The inverting thrombectomy apparatus and expandable scraper may be deployed through a sheath hub 1933.

The outer catheter 1905 may include multiple elements (control wires, shafts, etc.) to manipulate and/or articulate objects at the distal end. In particular, the methods and apparatuses for controlling a catheter described herein may be useful for preventing undesirable movement in an outer catheter of the mechanical thrombectomy apparatus. For example, a medical procedure may require the clinician to move portions of the catheter separately. A mechanical thrombectomy apparatus may have an inner catheter 1905 having a lumen holding an inner shaft 1913 (e.g., puller). Manipulation of a tool may require that the inner shaft move with respect to the outer catheter. In some cases, providing the relative motion may require holding the outer catheter while at the same time moving the inner shaft. However, the clinician may also need to guide the catheter at the same time, making control of the catheter-based system difficult.

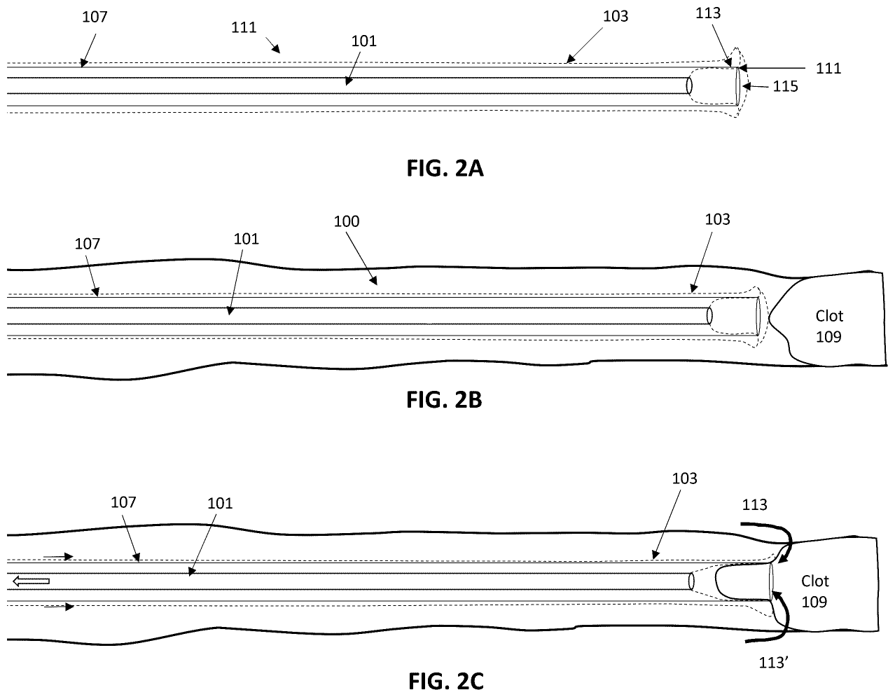

In general, an inverting tube apparatus (also referred to herein as “mechanical thrombectomy apparatus” or “inverting thrombectomy apparatus”) may be configured to remove material, such as clot, using a length of inverting tube, as shown in FIGS. 2A-2C. The apparatuses and methods of using them described herein may be used within the vasculature, including the neurovasculature and the peripheral vasculature.

For example, FIG. 2A illustrates an example of an inverting thrombectomy apparatus 100, such as described in U.S. patent application Ser. No. 15/496,570, and in U.S. Pat. No. 9,463,035. The apparatus includes an inversion support catheter 107 and a flexible tube 103 that extends over the outer surface of the inversion catheter. The flexible tube may be referred to as a tractor tube (or flexible tractor tube) and may be attached at one end region to a puller 101, which may be pull wire or pull tube (e.g., catheter), e.g., at the distal end region of the puller. In some examples, the flexible tube may be attached proximal to the distal end of the puller (e.g. between 1 mm and 50 mm from the distal end, between 1 mm and 40 mm, between 1 mm and 30 mm, greater than 5 mm, greater than 10 mm, greater than 20 mm, greater than 30 mm, etc. from the distal end of the puller). Pulling the puller proximally inverts the flexible tube over the distal end opening 111 of the inversion support catheter to capture and remove a material (such as a clot) in the vessel lumen, as shown in FIGS. 2B and 2C. In operation, the amount of the material that may be captured corresponds to the length of the flexible tube.

In FIG. 2B the inverting tractor mechanical thrombectomy apparatus 100 is shown deployed near a clot 109. In the deployed configuration the puller 101 (shown here as a puller micro catheter, alternatively the puller may be a wire) is held within an elongate inversion support catheter 107 so that the flexile tractor tube 103 extends from the end of the puller 101 and expands toward the inner radius of the elongate inversion support catheter 107; at the distal end opening 111 of the elongate inversion support catheter the tractor tube inverts over itself and extends proximally in an inverted configuration over the distal end of the elongate inversion support catheter. As shown in FIG. 2C, by pulling the puller proximally, the tractor tube rolls 113, 113′ and everts over the distal end opening of the elongate inversion support catheter, drawing the adjacent clot into the elongate inversion support catheter, as shown.

FIG. 2A the elongate inversion support catheter is an elongate tube having a distal end that has the same size inner diameter as the proximal length of the inversion support catheter. In some examples the distal end of the inversion support catheter may be funnel-shaped (or configured to expand into a funnel shape, see, e.g. FIGS. 2A-2B). In FIGS. 2A-2C, the inversion support catheter 107 is shown positioned between the tractor tube (e.g., flexible tube 103) and the puller 101 so that the flexible tube can be pulled proximally by pulling on the puller and rolling the flexible tube into the elongate inversion support catheter so that it inverts. The portion of the flexible tube that is inverted over the distal end of the elongate inversion support catheter has an outer diameter that is greater than the outer diameter of the elongate inversion support catheter. The flexible tube may be biased so that it has a relaxed expanded configuration with a diameter that is greater than the outer diameter (OD) of the elongate inversion support catheter; in addition, the flexible tube may also be configured (e.g., by heat setting, etc.) so that when the flexible tube is everted and rolled over the distal end opening into the elongate inversion support catheter, the outer diameter of the flexible tube within the elongate inversion support catheter has an outer diameter that is about y times (y fold) the inner diameter of the elongate inversion support catheter (e.g., where y is greater than 0.1×, 0.5×, 0.6×, 0.7×, 0.75×, 0.8×, 0.9×, 1×, etc. the inner diameter, ID, of the elongate inversion support catheter. This combination of an un-inverted diameter of the flexible tube of greater than the diameter of the OD of the elongate inversion support catheter and an inverted diameter of the flexible tube of greater than, e.g., 0.7× the ID of the elongate inversion support catheter is surprisingly helpful for preventing jamming of the apparatus, both when deploying the apparatus and when rolling the flexible tube over the distal end opening of the elongate inversion support catheter to grab a clot. The flexible tube may be expandable and may be coupled to the puller as shown. In some examples the flexible tube and the puller may comprise the same material, but the flexible tube may be more flexible and/or expandable, or may be connected to elongate puller (e.g., a push/pull wire or catheter). As mentioned above, the puller may be optional (e.g., the flexible tube may itself be pulled proximally into the inversion support catheter).

In FIG. 2C the clot may be drawn into the elongate inversion support catheter by pulling the flexible tube proximally and inverting the inverting tube 103 of the thrombectomy apparatus 100 into the distal end of the elongate inversion support catheter 107, as indicated by the arrows 113, 113′ showing pulling of the inner portion of the flexible tube, resulting in rolling the flexible tube over the end opening of the catheter and into the catheter distal end and inverting the expandable distal end region so that it is pulled into the catheter, shown by arrows. The end of the flexible tube outside of the catheter may be loose relative to the outer wall of the catheter.

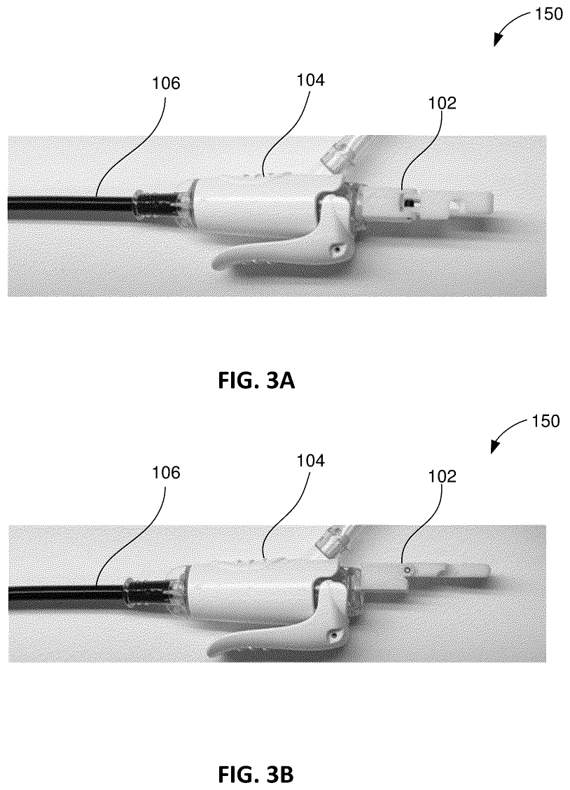

In general, the mechanical thrombectomy apparatuses described in FIGS. 2A-2C may be used with an introducer sheath (as shown in FIG. 1), as well as any of the introducer lock apparatuses as described in greater detail below. For example, FIGS. 3A-3B shows one example of an introducer lock apparatus coupled with an introducer sheath.

Examples of the subject matter described in this disclosure may be used to temporarily grip or restrain a portion of the outer catheter enabling the clinician greater control of the mechanical thrombectomy apparatus. Some examples may include an introducer lock apparatus that may be removably attached to an introducer sheath. The introducer lock apparatus may include a first body hingedly coupled to a second body. A catheter may pass through the introducer sheath and a clamping channel of the second body. As the catheter moves proximally with respect to the introducer sheath, the clamping channel and the second body may increase engagement with an outer portion of the catheter thereby clamping the catheter.

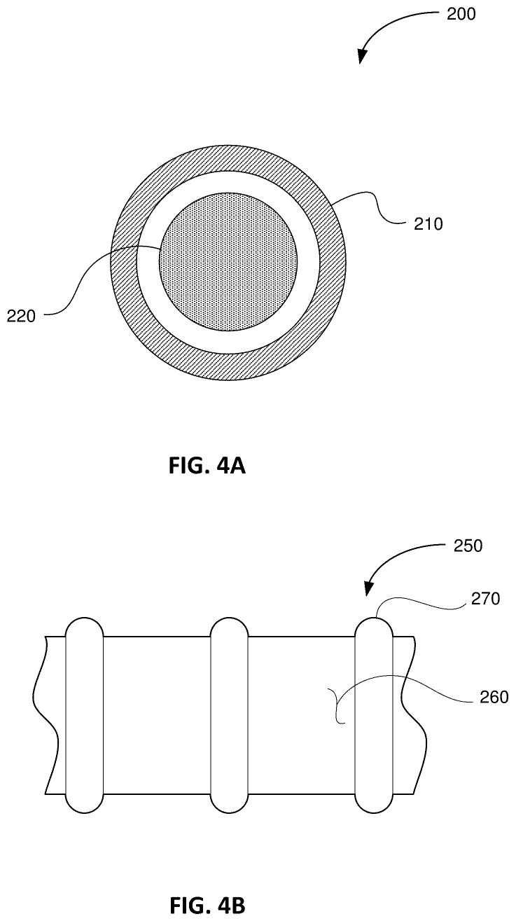

FIG. 3A shows a first view of a catheter controlling system 150, in accordance with some examples. The catheter controlling system 150 may include an introducer lock apparatus 102, an introducer sheath 104, and a sheath 106 (the sheath is the distal portion of the introducer sheath in this example). In some examples, the introducer lock apparatus 102 may be removably attached to the introducer sheath 104. For example, the introducer lock apparatus 102 may include one or more male locking lugs (not shown) that engage with one or more female channels (also not shown) in the introducer sheath 104. In some examples, the male locking lugs and the female channels may enable the introducer lock apparatus 102 to be attached with a quarter-turn (e.g., about ninety degrees) of rotation of the introducer lock apparatus 102 with respect to the introducer sheath 104. For example, the introducer lock apparatus 102 may be rotated one quarter-turn clockwise with respect to the introducer sheath 104 to removably attach the introducer lock apparatus 102 to the introducer sheath 104. The sheath 106 may be coupled to the introducer sheath 104. In some examples, the sheath 106 may receive and guide a catheter (not shown) into a blood vessel or other body lumen of a patent. The catheter may be part of a mechanical thrombectomy apparatus including an outer catheter that encloses at least an inner shaft as described in more detail below with respect to FIGS. 4A and 4B.

The introducer lock apparatus 102 may be initially inserted into the introducer sheath 104, as shown in the first view of a catheter controlling system 150. That is, the introducer lock apparatus 102 may not yet have been rotated a quarter-turn with respect to the introducer sheath 104. Thus, the introducer lock apparatus 102 may not yet be locked into the introducer sheath 104.

FIG. 3B shows a second view of the catheter controlling system 150, in accordance with some examples. As shown, the introducer lock apparatus 102 has been rotated clockwise a quarter-turn (about ninety degrees) with respect to the introducer sheath 104. In some examples, the quarter-turn revolution may allow the male locking lugs of the introducer lock apparatus 102 to fully engage with the female channels of the introducer sheath 104.

FIG. 4A shows a cross sectional view of a mechanical thrombectomy apparatus 200 as described above in FIGS. 1 and 2A-2C, that may include an outer catheter 210 and an inner shaft 220 (e.g. puller). The inverting tube (e.g., mesh, woven, etc.) is not shown. As shown, the outer catheter 210 may completely surround the inner shaft 220. The outer catheter 210 may move independently from the inner shaft 220. However, fitment between the outer catheter 210 and the inner shaft 220 may be such that there may exist a non-insubstantial friction between the two. In some examples, the mechanical thrombectomy apparatus 200 may include any feasible number of elements surrounded by the outer catheter 210. For example, the outer catheter 210 may surround two or more shafts, wires, tendons, or the like.

In some cases, one or more mechanical devices may be attached to and be operated by a distal end of the mechanical thrombectomy apparatus 200. For example, differential movement between the outer catheter 210 and the inner shaft 220 may operate a distally coupled mechanical device. Operation of the mechanical device may require that differential forces be applied between the outer catheter 210 and the inner shaft 220.

Thus, moving the inner shaft 220 with respect to the outer catheter 210 may require holding or gripping the outer catheter 210 while applying force to the inner shaft 220. In some cases, gripping the outer catheter 210 may be necessary to overcome friction or operate a mechanical device with the inner shaft 220. If the mechanical thrombectomy apparatus 200 is inserted into the introducer sheath 104 and the sheath 106 of FIGS. 3A and 3B, the introducer lock apparatus 102 may be used to grip, hold, and/or lock the outer catheter 210, particularly when articulating or moving the inner shaft 220. In some examples, the introducer lock apparatus 102 may preferentially grip the outer catheter 210 in one direction with respect to the introducer sheath 104.

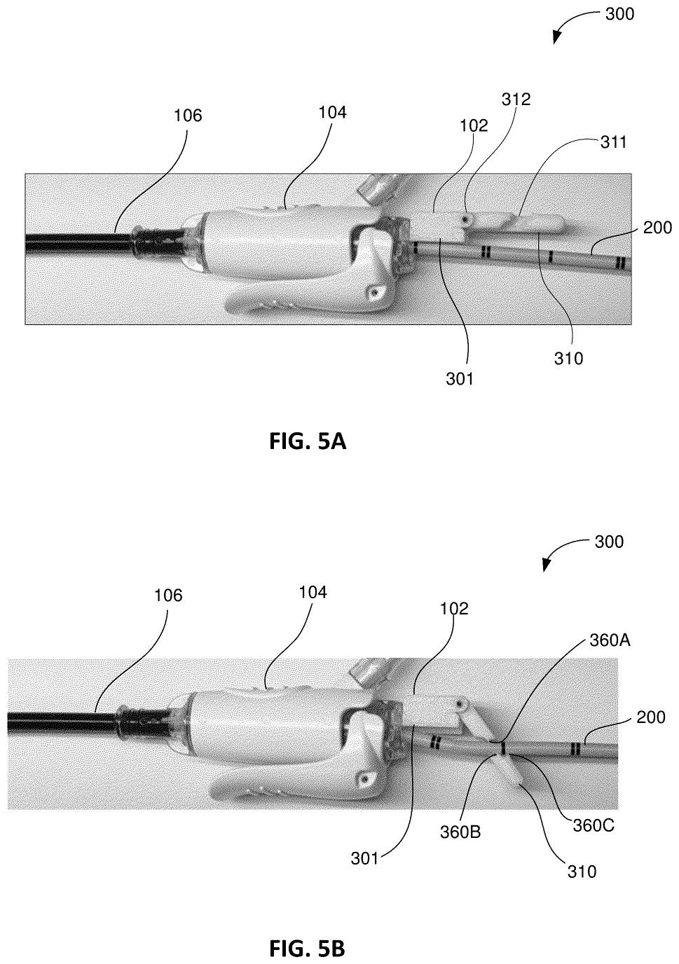

FIG. 4B shows a side view of a catheter 250, in accordance with some examples. The catheter 250 includes an outer catheter 260 and stops 270. The outer catheter 260 may be an example of the outer catheter 210 of FIG. 4A. Furthermore, the outer catheter 260 may enclose or surround any number of shafts, wires, tendons, or the like. The catheter 250 may include stops 270 that may be molded as part of the outer catheter 260. In some other examples, the stops 270 may be coupled or attached to the outer catheter 260. The stops 270 may be slightly larger (in diameter, for example) than the outer catheter 260. Furthermore, the number and spacing of the stops 270 may vary based on implementation. As will be discussed with respect to FIG. 5B, the stops 270 may enable the introducer lock apparatus 102 to better grip the outer catheter 260.

FIG. 5A shows a first view of a catheter controlling system 300, in accordance with some examples. The catheter controlling system 300 may include the introducer lock apparatus 102, the introducer sheath 104, and the sheath 106 of FIGS. 3A and 3B as well as the mechanical thrombectomy apparatus 100 of FIG. 2. As shown, the introducer lock apparatus 102 may be attached to the introducer sheath 104 and may be turned a quarter-turn so that the introducer lock apparatus 102 is attached and/or coupled to the introducer sheath 104. The mechanical thrombectomy apparatus 200 may be inserted through the introducer sheath 104, the introducer lock apparatus 102, and the sheath 106.

The introducer lock apparatus 102 may include a first body 301 and a second body 310. The first body 301 may include male lugs (not shown) to engage with, attach, and/or couple to the introducer sheath 104. As shown, the first body 301 may be colinear and/or coplanar with respect to the mechanical thrombectomy apparatus 200. The second body 310 may include a clamping channel 311 to receive the mechanical thrombectomy apparatus 200.

The first body 301 may be hingedly attached to the second body 310. Thus, a hinge joint 312 that includes hinge pin may couple (e.g., hingedly couple) the first body 301 to the second body 310. In some examples, the introducer lock apparatus 102 may include a spring (not shown), such a spiral spring near or around the hinge pin to bias the second body 310 with respect to the first body 301. For example, the spring may bias a position of the second body 310 in the position shown and also allow the second body 310 to pivot to receive the mechanical thrombectomy apparatus 200 in the clamping channel 311. In some cases, the hinge joint 312 may allow the second body 310 to pivot in a plane perpendicular to an axis or centerline of the mechanical thrombectomy apparatus 200.

FIG. 5B shows a second view of the catheter controlling system 300. In this view, the second body 310 has been pivoted toward the mechanical thrombectomy apparatus 200 such that the mechanical thrombectomy apparatus 200 has been received (positioned) within the clamping channel 311. In some examples, the clamping channel 311 may include one or more teeth 360A-360C that may engage with the outer catheter 210 of the mechanical thrombectomy apparatus 200.

In some examples, the second body 310 may pivot approximately 135 degrees relative the first body, where the second body is fully extended when the second body is in the fully extended position (180 degrees from the first body). Alternatively, if the angle is measure as the change in angle from the fully extended position, the first body is forty-five degrees with respect to the first body 301. This first angle (135 degrees) may be the angle at which the clamping channel has an apparent diameter through the clamping channel in the first axis that is at a maximum when the second body is bent at a first angle relative to the first body to receive the mechanical thrombectomy apparatus 200. Thus, the clamping channel 311 may be configured to accept a diameter of the outer catheter 210 when pivoted at about forty-five degrees with respect to the first body 301.

As the inner shaft 220 is moved proximally with respect to the introducer sheath 104, the outer catheter 210 may also be pulled proximally. Motion of the outer catheter 210 may be due to friction, forces required to operate a mechanical device coupled to a distal end of the mechanical thrombectomy apparatus 200, or the like. (For reference, the proximal direction is to the right and the distal direction is to the left in FIGS. 5A and 5B.) Notably, as the outer catheter 210 tries to move in the proximal direction, the outer catheter 210 increases engagement with the clamping channel 311 (and in some cases the teeth 360A-360C) in the second body 310. However, since the second body 310 is coupled to the first body 301, and since the first body 301 is coupled to the introducer sheath 104, the outer catheter 210 is prevented from moving with respect to the introducer sheath 104 in response to proximal motion of the inner shaft 220.

In some examples, one or more stops (not shown) may be molded or attached to the outer catheter 210. The stops may be larger than the outer catheter 210 and may increase engagement with the clamping channel 311 and/or the teeth 360A-360C compared to just the outer catheter 210.

During use, if the mechanical thrombectomy apparatus 200 is moved in a distal direction with respect to the introducer sheath 104, the second body 310 may also move distally relaxing the grip on the mechanical thrombectomy apparatus 200. Thus, the mechanical thrombectomy apparatus 200 may be preferentially allowed to move distally, while preferentially restricted from moving proximally.

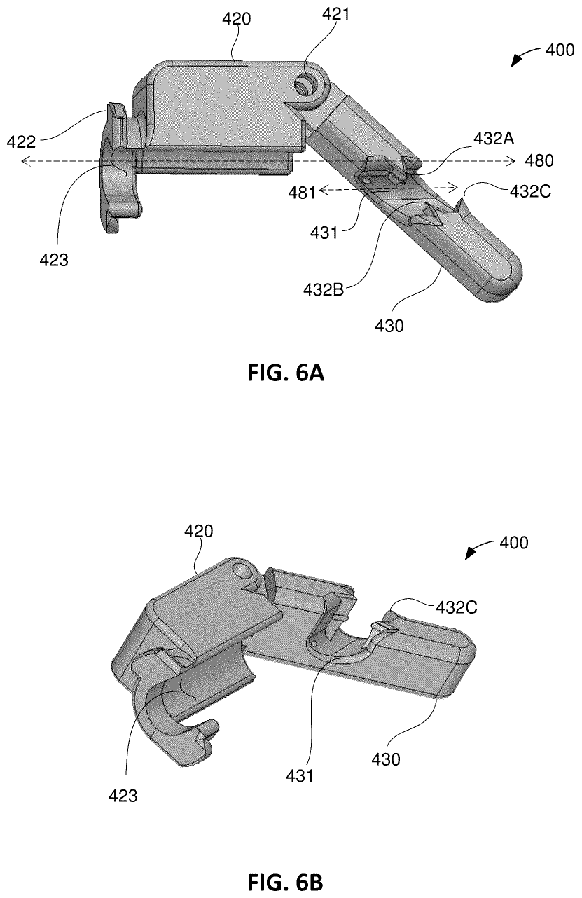

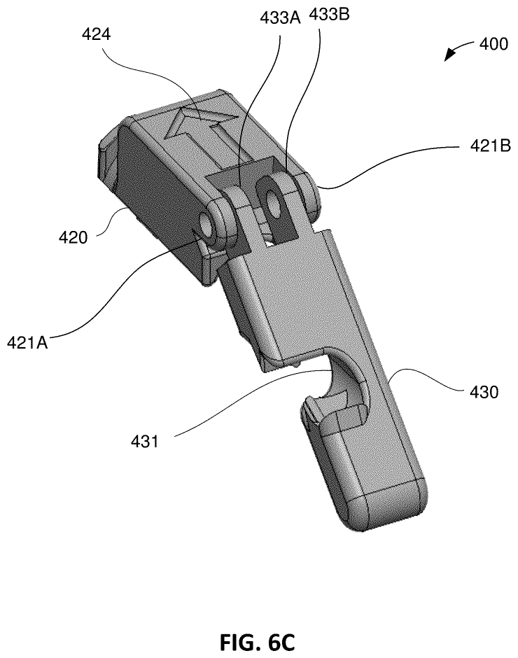

FIG. 6A shows a first view of an introducer lock apparatus 400, in accordance with some examples. The introducer lock apparatus 400 may include a first body 420 and a second body 430. The first body 420 may be an example of the first body 301 and the second body 430 may be an example of the second body 310 of FIGS. 5A and 5B.

In FIG. 6A, the first body includes the catheter channel 423 that is open on one side. The catheter channel includes a long axis 480 extending along the length of the catheter channel. The second body 430 includes a clamping channel 431 formed at an angle through the second body. In FIG. 6A, with the second body bent at an approximate 135 degree angle relative to the first body by bending at the hinge/hinge joint 421. In this configuration the clamping channel has an apparent diameter through the clamping channel in the first axis 481 (that is parallel to the long axis of the catheter seat 480). The apparent diameter is the diameter seen by the outer catheter as it is passed (in line with the catheter channel) through the clamping channel; in FIG. 6A the clamping channel is at a maximum apparent diameter when the second body is bent at a first angle (e.g., approximately 120, 135, 140 etc.) relative to the first body.

The first body 420 may include a hinge joint 421, locking lugs 422, and a catheter channel 423. The hinge joint 421 may enable the first body 420 to hingedly couple and pivot with respect to the second body 430. For example, the hinge joint 421 may enable the first body 420 to pivot about a hinge pin (not shown) on an axis perpendicular to an axis of a catheter, such as the mechanical thrombectomy apparatus 200 (also not shown). The catheter channel 423 may enable the catheter to pass proximate to the first body 420 without binding or any other impediment.

The locking lugs 422 may be male lugs that are configured to fit and mate with a female channel within the introducer sheath 104. Notably, the locking lugs 422 may include a gap or opening that advantageously allows attachment of the introducer lock apparatus 400 to the introducer sheath 104 after a catheter has been inserted in the introducer sheath 104 and/or sheath 106.

The second body 430 may also include a hinge joint (hidden in this view), a clamping channel 431, and teeth 432A-432C. The second body 430 may be coupled to the hinge joint 421 with a hinge pin thereby enabling the second body 430 to pivot about the hinge pin with respect to the first body 420. For example, the hinge joint may enable the second body 430 to pivot about a hinge pin on an axis perpendicular to an axis of the mechanical thrombectomy apparatus 200.

The clamping channel 431 may generally be shaped as a three-sided slot within the second body 430. Two of the sides of the clamping channel 431 may be approximately parallel with each other and disposed at approximately a forty-five degree angle with respect to a surface of the second body 430. A plurality of teeth 432A-432C may be disposed within the clamping channel 431. Although three teeth 432A-432C are shown, in other examples the clamping channel 431 may include any feasible number of teeth. In some examples, at least one of the teeth 432A-432C may be perpendicular to the parallel surfaces of the clamping channel 431. For example, tooth 432C may be relatively perpendicular with respect to the parallel surfaces of the clamping channel 431. The teeth 432A-432C may enable the second body 430 to grip the outer catheter 210 of the mechanical thrombectomy apparatus 200. In some examples, the teeth 432A-432C may grip one or more stops molded or attached to the outer catheter 210. The tooth that projects out of the clamping channel may be referred to as a locking tooth 432C.

FIG. 6B shows a second view of the introducer lock apparatus 400, in accordance with some examples. The second view includes the first body 420 and the second body 430 of the introducer lock apparatus 400. The second view depicts the catheter channel 423 in the first body 420. In some examples, the catheter channel 423 may run the length of the first body 420. The second view also shows the clamping channel 431 on the second body 430.

FIG. 6C shows a third view of the introducer lock apparatus 400, in accordance with some examples. The third view includes the first body 420 and the second body 430 of the introducer lock apparatus 400. The third view depicts hinge joints 433A and 433B of the first body 420. In some examples, the first body 420 may include any feasible number of hinge joints. The first body 420 may also include an arrow 424 to help guide the user during attachment of the introducer lock apparatus 400 to an introducer sheath.

The second body 430 may include hinge joints 433A and 433B. In some examples, the second body 430 may include any feasible number of hinge joints. Another view of the clamping channel 431 is also shown.

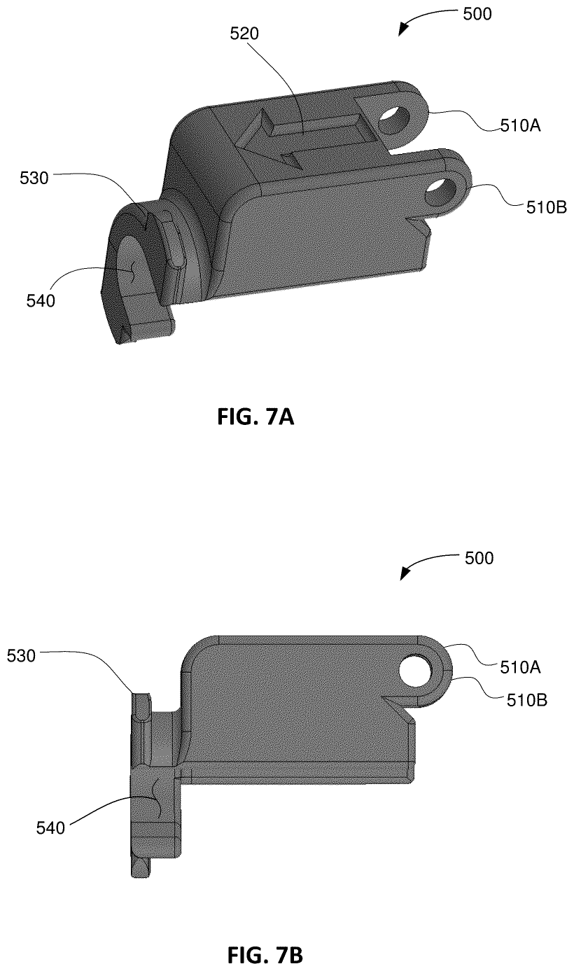

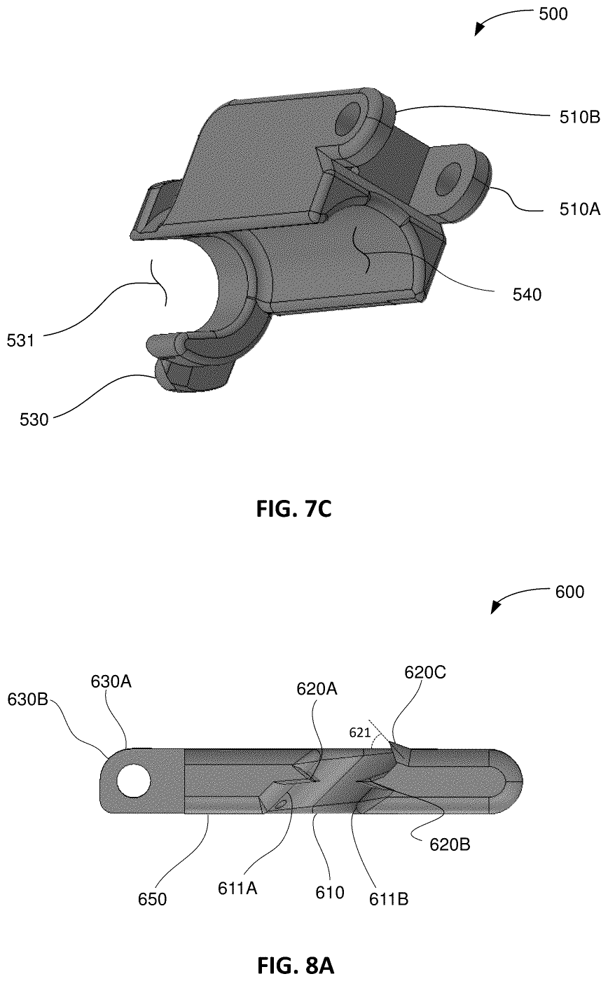

FIG. 7A shows a first view of a first body 500 of the introducer lock apparatus 400 of FIGS. 6A-6C. The first body 500 may include hinge joints 510A and 510B, an arrow 520, locking lugs 530, and a catheter channel 540. The hinge joints 510A and 510B may be examples of the hinge joints 421A and 421B of FIGS. 6A-6C. In some examples, the hinge joints 510A and 510B may receive a hinge pin.

The arrow 520, which may be an example of the arrow 424 of FIG. 6C, may assist the user during insertion of the first body 500 into an introducer sheath. The locking lugs 530 may be examples of the locking lugs 422 of FIG. 6A. Similarly, the catheter channel 540 may be an example of the catheter channel 423 of FIGS. 6A and 6B.

FIG. 7B shows a second view of the first body 500 of the introducer lock apparatus 400 of FIGS. 6A-6C. The second view depicts additional views of the hinge joints 510A and 510B, the locking lugs 530, and the catheter channel 540. FIG. 7C shows a third view of the first body 500 of the introducer lock apparatus 400 of FIGS. 6A-6C. The third view depicts additional views of the hinge joints 510A and 510B, the locking lugs 530, and the catheter channel 540. As shown, the locking lugs 530 may include a gap 531 that advantageously allows attachment of the introducer lock apparatus 400 to an introducer sheath after a catheter has been inserted in the introducer sheath 104 and/or sheath 106.

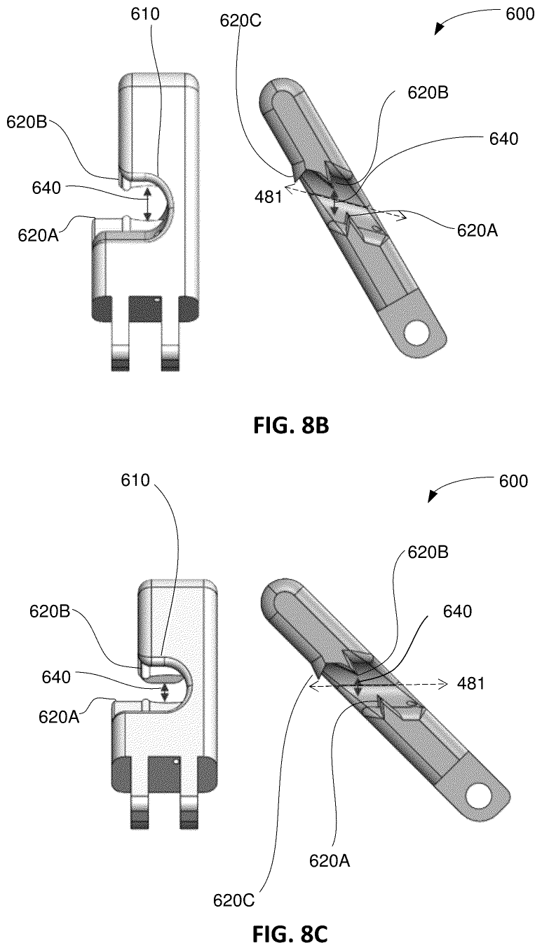

FIG. 8A shows a first view of a second body 600 of the catheter retainer lock 400 of FIG. 6A-6C. The second body 600 may include a clamping channel 610, teeth 620A-620C, and hinge joints 630A and 630B. In some examples, the hinge joints 630A and 630B may receive a hinge pin. The clamping channel 610 may be an example of the clamping channel 311 of FIGS. 5A and 5B and/or the clamping channel 431 of FIGS. 6A-6C. The teeth 620A-620C may be an example of the teeth 432A-432C of FIGS. 6A-6C.

The clamping channel 610 may include approximately parallel surfaces 613A and 613B. The teeth 620A-620C may be disposed on the parallel surfaces 611A and 611B. In particular, one or more of the teeth (620C) may be normal (e.g., perpendicular or ninety degrees) with respect to the parallel surfaces 611A and 611B. Furthermore, the parallel surfaces 611A and 611B may be disposed at approximately at an angle (e.g., between 30-50 degrees, between 35-55 degrees, about forty-five degrees, etc.) with respect to surface 650.

FIGS. 8B and 8C illustrate the apparent diameter through the clamping channel in two configurations. FIG. 8B shows a second view of the second body 600 of the catheter retainer lock 400 of FIGS. 6A-6C. The second view may illustrate the second body 600 positioned at a first angle with respect to the first body 500 (not shown). The teeth 620A-620C may form an opening 640. The apparent distance of the opening 640 may be based at least in part on the angle between the first body 500 and the second body 600. For example, as the second body 600 approaches a ninety-degree angle with respect to the first body 500, the opening 640 may become wider with respect to a catheter passing through the clamping channel 610. Conversely, as the second body 600 approaches a zero-degree angle with respect to the first body 500, the opening 640 may become narrower, with respect to the catheter passing through the clamping channel 610.

FIG. 8C shows a third view of the second body 600 of the catheter retainer lock 400 of FIG. 4. The opening 640 between teeth 620A-620C is shown. The third view shows the second body 600 at a lesser or decreased angle with respect to the first body 500 than the second body 600 of the second view. Thus, the third view shows the opening 640 being narrower than the opening 640 of the second view. In some examples, as the catheter engages with the teeth 620A-620C, the relative angle decreases between the first body 500 and the second body 600 causing the second body 600 to increase engagement with the catheter. Thus, proximal motion of a catheter in the clamping channel 610 may cause the second body 600 to pivot and have a decreased angle with respect to the first body 500. The decreased angle will decrease the opening 640 in the clamping channel 610 and cause the second body 600 to increase engagement with the catheter.

In this example, the locking tooth 620C is the tooth that extends out of the clamping channel and projects from an outer surface of the second body at a locking angle. As shown in FIG. 8A, the locking angle 621 may be about 45 degrees. In some examples the locking angle is between 35 and 55 degrees (e.g., between 40 and 50 degrees, etc.). The locking tooth may extend in the direction away from which the second body bends, e.g., away from the angle between the first body and the second body when the second body is engaged with the outer catheter, similar to that shown in FIGS. 6A and 6B. The locking tooth 620C is present on the upper surface of the second body (second arm).

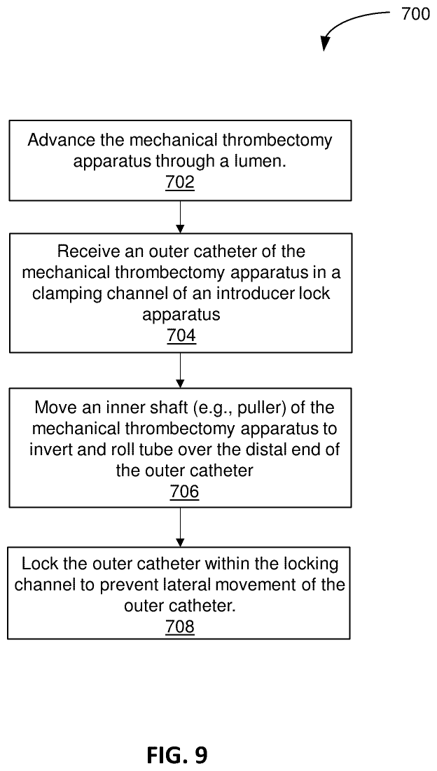

FIG. 9 is a flowchart depicting an example of one method 700 for controlling a mechanical atherectomy apparatus (system). Some examples may perform the operations described herein with additional operations, fewer operations, operations in a different order, operations in parallel, and some operations differently. The method 700 may be used to control movement of a mechanical atherectomy apparatus, particularly when motion is applied to one or more internal shafts of the outer catheter of the mechanical atherectomy apparatus.

In FIG. 9, the method 700 may begin as a mechanical atherectomy apparatus is advanced through a lumen in block 702. For example, a clinician may insert and guide an outer catheter of a mechanical atherectomy apparatus into a body lumen of a patient and adjacent to a clot.

Next, in block 704 an outer catheter is received in a clamping channel of an introducer lock apparatus. For example, an introducer lock apparatus may include a first body removably attached to an introducer sheath and a second body hingedly coupled to the first body. The second body may include a clamping channel that can receive the catheter, and in particular receive an outer catheter. In some examples, the clamping channel may be a slot disposed at approximately a forty-five degree angle with respect to an outer surface of the second body.

Next in block 706, the clinician may move an inner shaft of the mechanical atherectomy apparatus within the mechanical atherectomy apparatus in order to pull the inverting tube of the mechanical atherectomy apparatus into the outer catheter, rolling over the distal end opening of the outer catheter, but compressing the outer catheter, and potentially driving it proximally. For example, the clinician may move the inner shaft proximally with respect to the introducer sheath. As a result, the catheter in general, and the outer catheter in particular, may also move in a proximal direction.

Next in block 708, the clamping channel engages with the outer catheter in response to motion of the inner shaft. Since the second body (which includes the clamping channel) is coupled to the first body, and since the first body is coupled to the introducer sheath, motion of the catheter with respect to the introducer sheath may be reduced or limited as the clamping channel engages. For example, the clamping channel may increase a grip of the catheter as the inner shaft of the catheter moves in a proximal direction. In some examples, the clamping channel may include one or more teeth to engage and or grip the outer catheter. In other examples, the catheter may include one or more stops on the outer catheter to engage with the clamping channel.

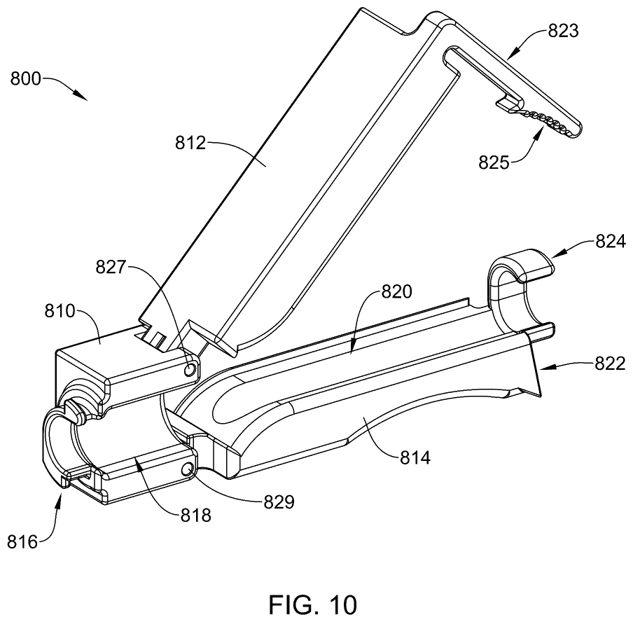

FIG. 10 illustrates another example introducer lock apparatus 800. The introducer lock apparatus 800 may include an attachment body 810 coupled to a first clamping arm 812 and a second clamping arm 814.

The attachment body 810 may include locking lugs 816 and a catheter channel 818. Additionally, the attachment body 810 be coupled to a first end of the first clamping arm 812 via a hinge joint, which may enable the first clamping arm 812 to pivot with respect to the attachment body 810. For example, the hinge joint may enable the first clamping arm 812 to pivot about a first hinge pin 827 on an axis perpendicular to an axis of a catheter channel 818. Similarly, attachment body 810 be coupled to a first end of the second clamping arm 814 via a hinge joint, which may enable the second clamping arm 814 to pivot with respect to the attachment body 810. For example, the hinge joint may enable the second clamping arm 814 to pivot about a second hinge pin 829 on an axis perpendicular to an axis of a catheter channel 818. It can be appreciated that each of the first clamping arm 812 and the second clamping arm 814 may each include a spring which may bias each of the first clamping arm 812 and the second clamping arm 814 in an open position. It can be further appreciated that the catheter channel 818 may enable a catheter shaft (e.g., introducer sheath, an accessory shaft, an outer catheter, guide sheath, etc.) to pass through the attachment body 810 without binding or any other impediment.

The locking lugs 816 may be male lugs that are configured to mate with a female channel within the manifold 830 of an accessory device (e.g., introducer sheath, guide sheath, guide catheter, etc.). The locking lugs 816 may include a gap or opening that allows attachment of the introducer lock apparatus 800 to attach to an introducer sheath after a catheter has been inserted in the introducer sheath and/or other accessory device (e.g., an outer sheath, guide sheath, guide catheter, etc.).

As discussed herein, the first clamping arm 812 may include a first end region which is hingedly attached to the attachment body 810. Similarly, FIG. 10 illustrates that the second clamping arm 814 may include a first end region which is hingedly attached to the attachment body 810. Accordingly, in addition to pivoting with respect to the attachment body 810, the first attachment arm 812 and the second attachment arm 814 may be pivot with respect to one another. Accordingly, the first attachment arm 812 and the second attachment arm 814 may pivot toward one another, thereby permitting the first attachment arm 812 and the second attachment arm 814 to cooperatively clamp onto a catheter shaft (e.g., an outer shaft of the thrombectomy system 200) extending through the attachment body 810.

FIG. 10 further illustrates that the second clamping arm 814 may include a clamping channel 820 (e.g., recess) extending along the length of the second clamping arm 814. The clamping channel 820 may be shaped to accommodate a catheter shaft which may be placed within the clamping channel 820 and extending along the length of the second clamping arm 814. It can be appreciated that a catheter shaft (e.g., an outer shaft) may extend along the channel 820 of the second clamping arm 814 and through the catheter channel 818.

FIG. 10 further illustrates that the second clamping arm 814 may include a catheter guide member 824 (e.g., cradle) positioned at a proximal end region of the clamping arm 814, whereby the catheter guide member 824 may be longitudinally aligned with the catheter channel 818. The catheter guide member 824 may be designed to support a catheter shaft secured thereto. For example, the catheter guide member 824 may include an arcuate shape (e.g., semi-circle, C-shaped, half-moon shape, etc.) designed to permit a catheter shaft (e.g., outer shaft) to be press-fit therein. The catheter guide member 824 may be designed to secure a catheter shaft at the proximal end of the second clamping arm 814, thereby aligning the catheter shaft with the catheter channel 818 of the attachment body 810.

As discussed herein, the first clamping arm 812 and the second clamping arm 814 may pivot relative to one another, thereby permitting the first attachment arm 812 and the second attachment arm 814 to cooperatively clamp onto a catheter shaft (e.g., an outer shaft of the thrombectomy system 200) extending through the attachment body 810. Further, FIG. 10 illustrates that the introducer lock 800 may include a locking mechanism designed to releasably secure the first clamping arm 812 to the second clamping arm 814. As illustrated in FIG. 10, the introducer lock 800 may include a locking arm 823 positioned along a proximal end region of the first clamping arm 812. The locking arm 823 may be designed to releasably engage (e.g., latch, snap, clip, etc.) onto a catheter lock receiving region 822 positioned along the proximal end region of the second clamping arm 814. The locking arm 823 may further include a release tab 825 designed to permit a user to unlatch the locking arm 823 from the catheter lock receiving region 822 of the second clamping arm 814. The release tab 825 may include a textured grip surface.

It can be appreciated from FIG. 10 that the locking arm 823 may be laterally offset relative to the longitudinal axis of the first clamping arm 812. FIG. 10 further illustrates that the catheter guide member 824 may also be laterally offset relative to the longitudinal axis of the second clamping arm 814. It can be appreciated that the position of the locking arm 823 and the position of the catheter guide member 824 may permit the first clamping arm 812 and the second clamping arm 814 to pivot relative to one another and cooperatively clamp onto a catheter shaft without the locking arm 823 interfering with the catheter guide member 824. In other words, as the first clamping arm 812 and the second clamping arm 814 to pivot toward one another and cooperatively clamp onto a catheter shaft, the locking arm 823 may be positioned aside the catheter guide member 824. Further, when the locking arm 823 is positioned aside the catheter guide member 824, the locking arm 823 may be releasably latched to the catheter lock receiving region 822, thereby releasably securing the first clamping arm 812 to the second clamping arm 814.

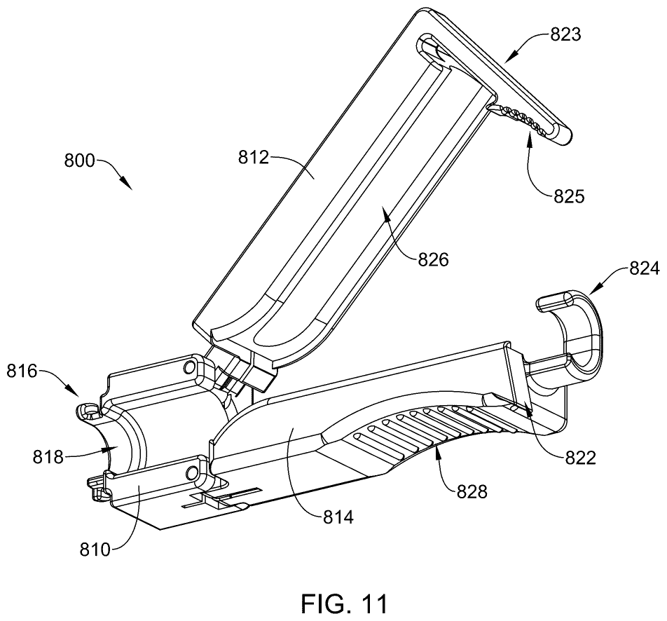

FIG. 11 illustrates another perspective view of the introducer lock 800, including the attachment body 810 coupled to a first clamping arm 812 and a second clamping arm 814. As discussed above, FIG. 11 illustrates the locking arm 823 which is designed to releasably engage (e.g., latch, snap, clip, etc.) onto a catheter lock receiving region 822 positioned along the proximal end region of the second clamping arm 814. In some examples (such as the example illustrated in FIG. 11), the locking arm 823 may include an angled projection (e.g., tooth) designed to snap over and releasably engage a surface of the catheter lock receiving region 822. It can be appreciated that the locking arm 823 may flex outward such that the locking arm 823 can releasably engage (e.g., snap over, latch, etc.) onto the catheter lock receiving region 822. To disengage the locking arm 823, a user may push the locking arm 823 outward and away from the catheter lock receiving region 822, thereby disengaging the locking arm 823 from the catheter lock receiving region 822.

FIG. 11 further illustrates that the first clamping arm 812 may include a clamping channel 826 extending along the length of the first clamping arm 812. The clamping channel 826 may generally be shaped to accommodate a catheter shaft which may be placed within the clamping channel 826 and extend along the length of the first clamping arm 812. It can be appreciated that a catheter shaft (e.g., an outer shaft) may both extend along the first clamping arm 812 and through the catheter channel 818.

FIG. 11 further illustrates that the second clamping arm 814 may include a finger grip region 828 positioned along an outer surface of the clamping arm 814. The finger grip region 828 may include a curved shape. Additionally, the finger grip region 828 may include a textured surface.

Further, each of the catheter channel 826 of the first clamping arm 812 and the catheter channel 820 of the second clamping arm 814 may include a pad adhered to a surface thereof. For example, each of the catheter channel 826 of the first clamping arm 812 and the catheter channel 820 of the second clamping arm 814 may include a silicone pad disposed along the respective channel, whereby the silicone pad may change its shape (e.g., stretch, deform, etc.) to adapt to a catheter shaft extending along each of the first clamping arm 812 and the second clamping arm 814. In other words, silicone pads positioned along the catheter channel 826 and the catheter channel 820 may stretch and grip a catheter shaft positioned between the first clamping arm 812 and the second clamping arm 814 when the first clamping arm 812 and the second clamping arm 814 are releasably attached to one another.

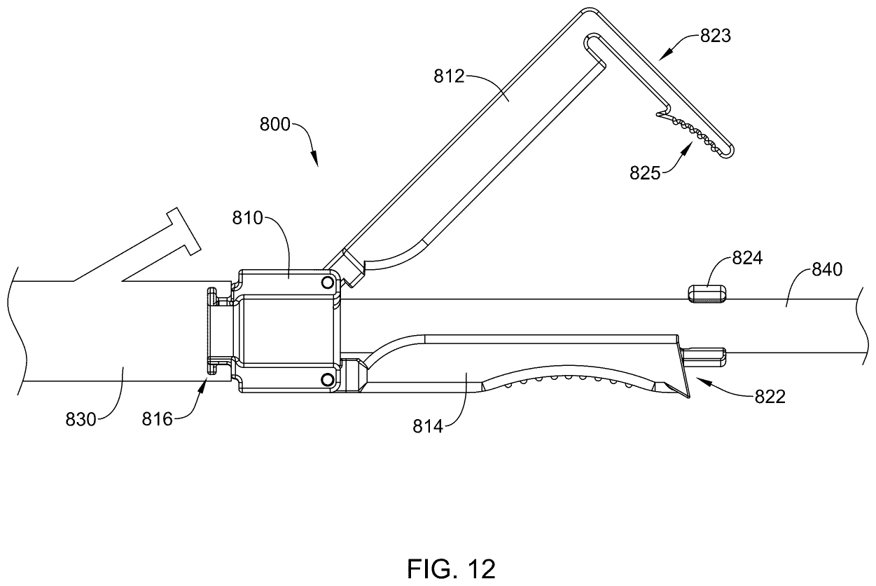

FIG. 12 illustrates the introducer lock apparatus 800 attached to a manifold 830 of an example introducer sheath. The manifold 830 of the introducer sheath may be similar to the introducer sheath 104 disclosed herein. Accordingly, FIG. 12 illustrates that the locking lugs 816 of the attachment body 810 may engage with the manifold 830 of the introducer sheath to couple the introducer lock 800 to the introducer sheath. It can be appreciated that the locking apparatus 800 may be used with any accessory medical device, including, but not limited to introducer sheaths, guide sheaths, guide catheters, etc.

FIG. 12 further illustrates a shaft 840 (e.g., outer shaft) positioned along the catheter channel 820 of the second clamping arm 814. The shaft 840 may be a component of a mechanical thrombectomy apparatus (such as an outer shaft of the mechanical thrombectomy apparatus 200 discussed herein). It can be appreciated that the introducer locking apparatus 800 illustrated in FIG. 12 may be in an open, unlocked configuration, whereby the first clamping arm 812 is pivoted away from the second clamping arm 814. As discussed herein, it can be appreciated that a spring may bias the first clamping arm 812 in the open configuration whereby it is spaced away from the first clamping arm 814 (as shown in FIG. 12) when not releasably attached to the second clamping arm 814.

FIG. 12 further illustrates that the shaft 840 may extend through the attachment body 810 and into a lumen of the manifold 830. Additionally, FIG. 12 illustrates that the shaft 840 may be releasably secured within the catheter guide member 824, which aligns the shaft 840 with the catheter channel 818 (shown in FIG. 10) of the attachment body 810.

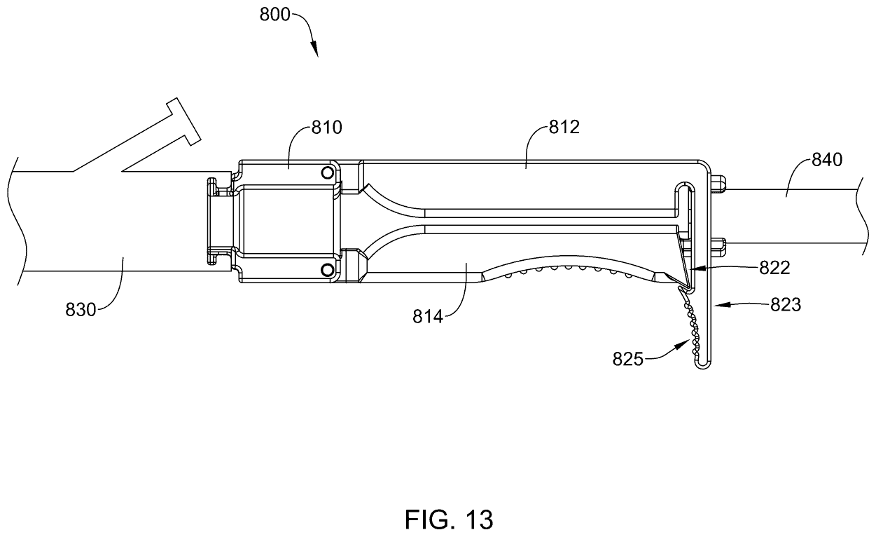

FIG. 13 illustrates the introducer locking apparatus 800 in a locked configuration, whereby the first clamping arm 812 has been pivoted toward the first clamping arm 814, whereby the shaft 840 is positioned within the catheter channel 826 of the first clamping member 812 and the catheter channel 820 of the second clamping arm 814. Additionally, FIG. 13 illustrates that the locking arm 823 has been releasably engaged (e.g., secured, attached, latched, etc.) onto the catheter lock receiving region 822. As discussed herein, pads (e.g. silicone pads) disposed along the catheter channel 826 of the first clamping member 812 and the catheter channel 820 of the second clamping arm 814 may stretch to grip and secure the shaft 840 between the first clamping arm 812 and the second clamping arm 814, thereby substantially preventing the shaft 840 (e.g., an outer shaft) from shifting (e.g. migrating) relative to the manifold 830 of the introducer sheath (while another device is pulled and/or advanced through the shaft 840, for example). It can be appreciated that the locking arm 823 may be released from the catheter lock receiving region 822 via pushing the release tab 825 outward and away from the catheter lock receiving region 822.

Additionally, it can be appreciated the locking apparatus 800 may be designed to prevent the shaft 840 from being adversely bent (e.g., kinked, deformed, etc.) when clamped or secured between the first clamping arm 812 and the second clamping arm 814. As discussed herein, each of the first clamping arm 812 and the second clamping arm 814 may include a pad disposed thereon that may conform to the shape of the shaft 840, thereby providing both securement (e.g., grip) and cushion for the shaft 840 when clamped between the first clamping arm 812 and the second clamping arm 814 in the locked configuration. Further, it can be appreciated that the first clamping arm 812 and the second clamping arm 814 may be designed to maintain the shaft 840 in a substantially linear (e.g., unbent, unkinked) configuration when the shaft 840 is clamped between the first clamping arm 812 and the second clamping arm 814 in the locked configuration.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-15. (canceled)

16. A medical device lock for use with a thrombectomy system, comprising:
a body having a lumen extending therein;
a first clamping arm pivotably coupled to the body; and
a second clamping arm pivotably coupled to the body;
wherein the first clamping arm may be releasably attached to the second clamping arm to clamp a catheter shaft therebetween, wherein clamping the catheter between the first clamping arm and the second clamping arm maintains the catheter shaft in a substantially linear configuration.

17. The medical device lock of claim 16, wherein the first clamping arm is coupled to the body via a first hinge connection.

18. The medical device lock of claim 17, wherein the second clamping arm is coupled to the body via a second hinge connection.

19. The medical device lock of claim 18, wherein the first hinge connection includes a first spring configured to bias the first clamping arm in an unlocked configuration.

20. The medical device of claim 19, wherein the second hinge connection includes a second spring configured to bias the second clamping arm in the unlocked configuration.

21. The medical device lock of claim 16, wherein the lumen of the body is configured to permit the catheter shaft to extend therethrough.

22. The medical device lock of claim 16, wherein the first clamping arm includes a first clamping channel extending along a longitudinal axis of the first clamping arm, wherein the first clamping channel is configured to permit the catheter shaft to be disposed therein.

23. The medical device lock of claim 22, wherein the second clamping arm includes a second clamping channel extending along a longitudinal axis of the second clamping arm, wherein the second clamping channel is configured to permit the catheter shaft to be disposed therein.

24. The medical device lock of claim 23, wherein the first clamping channel, the second clamping channel or both the first clamping channel and the second clamping channel includes a silicone pad disposed thereon.

25. The medical device lock of claim 16, wherein the first clamping arm includes a locking member disposed along a proximal end region of the first clamping arm, and wherein the locking member is configured to releasably engage a portion of the second clamping arm.

26. The medical device lock of claim 16, wherein the second clamping arm includes a cradle disposed along a proximal end region of the second clamping arm, wherein the cradle is configured to accept the catheter shaft.

27. The medical device lock of claim 26, wherein the cradle is longitudinally aligned with the lumen of the attachment body.

28. The medical device lock of claim 16, further comprising a connector disposed along a distal end region of the body.

29. The medical device lock of claim 28, wherein the connector is configured to attach to a manifold of a medical introducer sheath.

30. The medical device lock of claim 28, wherein releasably attaching the first clamping arm to the second clamping arm to clamp a catheter shaft therebetween prevents the catheter shaft from translating relative to the manifold of the introducer sheath.

31. The medical device lock of claim 30, wherein the catheter shaft is an outer shaft of a thrombectomy apparatus.

32. A medical device lock for use with a thrombectomy system, comprising:
a body having a distal end region, a proximal end region and a lumen extending therein;
a first clamping arm coupled to the proximal end region of the body, wherein the first clamping arm is configured to pivot about a first spring-loaded hinge connection; and
a second clamping arm coupled to the proximal end region of the body, wherein the second clamping arm is configured to pivot about a second spring-loaded hinge connection;
wherein the first clamping arm may be releasably attached to the second clamping arm to clamp a catheter shaft between the first clamping arm and the second clamping arm in a locked configuration, and wherein clamping the catheter between the first clamping arm and the second clamping arm maintains the shaft in a substantially linear configuration.

33. The medical device of claim 32, wherein the first hinge connection includes a first spring configured to bias the first clamping arm in an unlocked configuration, and wherein the second hinge connection includes a second spring configured to bias the second clamping arm in the unlocked configuration.

34. The medical device lock of claim 32, wherein the first clamping arm includes a locking member disposed along a proximal end region of the first clamping arm, and wherein the locking member is configured to releasably engage a portion of the second clamping arm in the locked configuration.

35. A method of securing a portion of a thrombectomy system relative to an introducer, the method comprising:
positioning the introducer within a body vessel of a patient, the introducer including a shaft coupled to a manifold;
securing a medical device lock to the manifold of the introducer, the medical device lock including:
a body having a lumen extending therein;
a first clamping arm pivotably coupled to the body; and
a second clamping arm pivotably coupled to the body;
wherein the first clamping arm may be releasably attached to the second clamping arm; and
advancing an outer catheter of the thrombectomy system through the lumen of the medical device lock body, through the manifold of the introducer and into the lumen of the introducer;
attaching the first clamping arm to the second clamping arm such that the outer catheter is clamped between the first clamping arm and the second clamping arm, and wherein clamping the outer shaft between the first clamping arm and the second clamping arm prevents the outer catheter from translating relative to the introducer.
